(19) United States
(12) Patent Application Publication
(10) Pub. No.: US 2022/0288251 A1
(43) Pub. Date: Sep. 15, 2022

(54) HANDHELD GERMICIDAL LIGHT TOOL WITH DISTANCE AND POWER FEEDBACK

(71) Applicant: CAO Group, Inc., West Jordan, UT (US)

(72) Inventor: Densen B. Cao, Sandy, UT (US)

(73) Assignee: CAO Group, Inc., West Jordan, UT (US)

(21) Appl. No.: 17/654,550

(22) Filed: Mar. 11, 2022

Related U.S. Application Data
(60) Provisional application No. 63/159,903, filed on Mar. 11, 2021.

(51) Int. Cl. A61L 2/10; A61L 2/28

(52) U.S. Cl. CPC A61L 2/10; A61L 2/28; A61L 2202/11; A61L 2202/14; A61L 2202/25

(57) ABSTRACT

A handheld disinfection tool utilizing UVC light may feature a UVC light source (104) coupled with sensors (109) to measure power density and distance (X) to a desired surface. Use of the sensors in conjunction with the UVC light source will allow a more continuous power density of UVC light to be present at the surface during a disinfecting procedure.

### CROSS-REFERENCES TO RELATED APPLICATIONS

This application claims priority to prior filed U.S. application 63/159,903, filed on Mar. 11, 2021, and incorporates the same by reference in its entirety herein.

### FIELD OF THE INVENTION

The present invention relates to the field of disinfection and more particularly relates to a handheld tool emitting UVC energy for the purpose of local surface disinfection.

### BACKGROUND OF THE INVENTION

Controlling bacteria and viruses in professional offices, homes, and other public areas is particularly important in the advancement of human/animal health. Disinfecting the working environment, particularly high-contact surfaces, is critical for medical, dental, veterinary, and other fields to prevent cross-contamination and infections. Currently, surface disinfection requires the use of surface disinfectants which must be applied and removed, usually after a prescribed “dwell time” wherein the disinfectant must sit on the surface and kill microbes before it is removed. During such dwell times, the disinfectant usually must remain in an aqueous state. While this strategy can be effective, it is only moderately so if proper protocols are followed, and it is time consuming. Human error and impatience tend to reduce the effectiveness of surface disinfectants.

Another strategy is the use of ultraviolet light, particularly in the “UVC” band (220 nm to 300 nm). These UVC light sources have been proven to be an effective tool in the disinfection of surfaces. One critical aspect of using UVC light for the disinfection of surfaces is to maintain an effective UV dose, or fluence, (measured in millijoules/square centimeter (mJ/cm² or mW*s/cm²)). Average bacteria and viruses will see at least a 99.9% reduction once exposed to 50 mJ/cm², though some may require a larger dose. The UV dose is achieved by applying an appropriate power density of the light for a requisite time period so that its energy may kill pathogens on a surface. Lighting power density (“LPD”), measured in milliwatts/square centimeter (mW/cm²), will vary inversely with the square of the distance between the UVC source and the targeted surface, so small variations in distance can yield significant variations in LPD. Lower LPD values require higher exposure times to compensate for the lower power. When an individual uses a hand-held device for UVC disinfection, it can be difficult to maintain effective LPDs given that individuals may hold a UVC light source from a surface at irregular distances. This variation will not only appear between different individuals but a single individual will also have inherent variation. The present invention is a handheld tool that provides UVC light for the purpose of disinfecting contact surfaces in a workplace, home, or other environment while maintaining control of the LPD of the UVC output by monitoring distance between the UVC source and the target surface.

### SUMMARY OF THE INVENTION

In view of the foregoing disadvantages inherent in the known types of disinfectant strategies, an improved UVC light source for the purpose of disinfection will provide a warning if distance to the target surface and the UVC light source exceeds a maximum limit. This new UVC light source may provide a handheld tool that meets the following objectives: that it be inexpensive to manufacture, that it be intuitive in its use, that it may provide UVC light at a sufficient power density to disinfect a surface within a brief period. As such, a new and improved handheld disinfection tool may comprise a handheld body in which a UVC light source, such as one or more UVC Light Emitting Diodes (“LEDs”), is mounted, complete with controls for the activation and adjustment of the UVC light source for disinfecting purposes, and a distance sensor to measure the distance from the UV light source to the surface and help control power output to accomplish these objectives.

The more notable features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in several ways. Also, it is to be understood that the phraseology and terminology employed herein are for description and should not be regarded as limiting.

As such, those skilled in the art will appreciate that the conception, upon which this disclosure is based, may readily be utilized as a basis for the designing of other structures, methods, and systems for carrying out the several purposes of the present invention. It is important, therefore, that the claims be regarded as including such equivalent constructions as far as they do not depart from the spirit and scope of the present invention.

### BRIEF DESCRIPTION OF THE DRAWINGS

To describe the way the above-recited and other advantages and features of the invention can be obtained, a more particular description of the invention briefly described above will be rendered by reference to specific example embodiments thereof which are illustrated in the appended drawings. Understanding that these drawings depict only typical embodiments of the invention and are therefore not to be considered as limiting of its scope, the invention will be described and explained with additional specificity and detail using the accompanying drawings.

FIG. 1 is a schematic view of one embodiment of the invention utilizing an LED.

FIG. 2 is a sectional schematic view of the embodiment of the invention shown in FIG. 1.

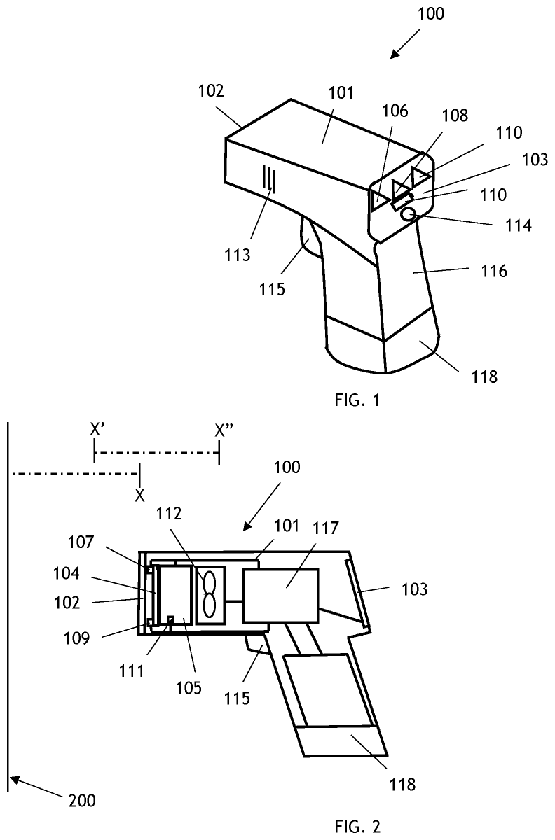

### DETAILED DESCRIPTION OF THE PREFERRED EMBODIMENT

With reference now to the drawings, a preferred embodiment of the handheld germicidal light tool is herein described. It should be noted that the articles “a,” “an,” and “the,” as used in this specification, include plural referents unless the content clearly dictates otherwise.

With reference to the Figures, a handheld tool (100) features an LED as a UVC light source where (101) is the casing, which is illustrated as handheld or gun-type. The shape of device can be other shapes—such as linear, like a cylindrical flashlight. A UVC light source exit, ideally with a UVC transparent window (102), is aimed towards a surface (200) to be disinfected and defines a front of the tool (100). There is a display panel (103) on a back surface of the device which contains indicators and a safety switch. A UVC light source, such as the depicted UVC LED (104) is mounted within the casing (101) and may be positioned upon a heat sink (105). Control circuitry (117) is also provided, connecting the power source, such as battery (118), sensors, control panel (103) with its indicators and controls, light source (104), control switch (115), and any other components in an operable manner.

The LPD of the emitted light decreases as the light source (104) moves away from the surface (200), therefore the handheld tool (100) includes a distance feedback system which includes a distance sensor (107), proximate the light exit (102), which measures the distance (X) of the light source from the surface (200) and a distance indicator (106). When the device (100) is in use, the distance sensor (107) will ideally continuously measure the distance between the surface and light source and its indicator (106) may activate to provide a warning if the light exit (102) is outside of a desired distance (X′<X<X″) from surface (200). The preferred distance range (X′ to X″) will be calculated from the strength of the initial output from light source (104) and the desired LPD necessary to ensure an effective UV dose within a desired amount of time. This range can include a distance where the light exit is proximate the surface (200) (X′=˜0 cm). This warning will notify the user and prompt said user to move the tool (100) to within the desired range (X′ to X″) and ensure that LPD at the surface (200) has the desired strength to kill microbes present thereon. Periodic measurements by the distance sensor (107) will not provide as ready of an indication when the tool (100) is outside the desired range, but will provide some indication of distance and still be preferred over the prior art.

Other indicators and controls may be provided on the display panel (103). A power indicator (108) may be coupled to a light power sensor (109), also proximate the light exit (102), to measure the light power output. If the light source has lower power output, the sensor (109) will send signal to indicator (108) to provide warning to users to check the light source. As some UVC light sources, such as LEDs, can have increased sensitivity to temperature, it may be desirable to provide a heat feedback system, including a heat indicator (110) and associated sensor (111) which may be used to indicate the temperature of light source. The heat sensor (111) is placed by the light source (104) to monitor the temperature of UVC source. If the heat exceeds a desired limit, the device may shut down and/or the indicator (110) may provide a warning before or during shut down. A fan (112) may be provided to provide air circulation within the casing (101) and cool the UVC source (104), particularly if the source is one or more UVC LEDs. This fan (112) may be manually or automatically controlled and may be controlled based upon the temperature of the UVC source (104). Vents (113) may be fashioned in the casing (101) to facilitate air flow.

A safety switch (114) may provide protection from accidental UVC light emission. In one embodiment switch (114) must be pushed first to actuate trigger switch (115), shown in the handle (116), to control the light emission. The safety protocol may be either require the user to push and hold down safety switch (114) while initially actuating trigger switch (115) or may be simply actuating safety switch (114) and then actuating the trigger switch (115) when ready.

Power for the system and its control circuit (117) may be provided by a battery pack (118) located in the bottom of the handle (116). Battery pack (118) may be replaceable, rechargeable, or both. A battery indicator (119) may be provided to indicate the battery level of the tool.

While the preferred UVC light source (104) is one or more UVC LEDs, other sources known in the art, such as mercury and amalgam lamp UVC light sources, may also be utilized. All the functions would be the same as described above when utilizing a mercury lamp instead of LEDs. However, mercury lamps are bulkier and more fragile compared to LEDs. Also, since these lamps contain mercury, they do present additional environmental issues. Therefore, the use of UVC LEDs is preferred, but the invention may be practiced using any UVC source.

Although the present invention has been described with reference to preferred embodiments, numerous modifications and variations can be made and still the result will come within the scope of the invention. For instance, various controls and features may be added to the control panel (103) to provide a customizable experience. Such features may include but are not limited to power density selection, variable distance settings and alarms, a timer, and fan controls. The described embodiments are to be considered in all respects only as illustrative and not restrictive. No limitation with respect to the specific embodiments disclosed herein is intended or should be inferred. Therefore, the scope of the invention is indicated by the appended claims rather than by the foregoing description. All changes which come within the meaning and range of equivalency of the claims are to be embraced within their scope.

What is claimed is:

1. An instrument to provide a beam of ultraviolet light, the instrument comprising:
an instrument casing having a beam exit port;
a UV light source residing in the casing to provide a desired light output beam through the beam exit port; and,
a distance feedback system, further comprising a distance sensor to measure a distance between the beam exit port and a desired target surface;
wherein the distance feedback system will utilize the distance sensor to determine the distance between the beam exit port and desired target and provide a warning if the distance is outside a set range.

2. The instrument of claim 1, the set range being calculated based upon a power of the beam generated by the UV light source and a desired lighting power density to be achieved at the surface.

3. The instrument of claim 1, the UV light source being at least one UVC emitting LED.

4. The instrument of claim 1, further comprising a heat feedback system.
